(19) United States
(12) Patent Application Publication
(10) Pub. No.: US 2005/0063942 A1
(43) Pub. Date: Mar. 24, 2005

(54) Methods for predicting sensitivity of tumors to arginine deprivation

(76) Inventors: Mike A. Clark, Lexington, KY (US); Charles Mark Ensor, Lexington, KY (US); Frederick Wayne Holtsberg, Nicholasville, KY (US)

Correspondence Address: WOODCOCK WASHBURN LLP, ONE LIBERTY PLACE, 46TH FLOOR, 1650 MARKET STREET, PHILADELPHIA, PA, 19103, US

(21) Appl. No.: 09/775,693

(22) Filed: Feb. 2, 2001

(51) Int. Cl. C12Q 1/68; G01N 33/574; A61K 38/19

(52) U.S. Cl. 424/85.1; 435/6; 435/7.23

(57) ABSTRACT

The present invention provides methods for determining which cancer patients are susceptible to arginine depletion therapy and methods for treating cancer. The present invention also provides methods for predicting the appropriateness of arginine deprivation therapy for a cancer patient. The methods generally comprise obtaining a tumor sample from the cancer patient and detecting the presence or absence of evidence of urea cycle enzyme expression in the tumor sample. The absence of evidence of urea cycle enzyme expression in the tumor sample is indicative of a cancer patient who is a candidate for arginine deprivation therapy, and the presence of evidence of urea cycle enzyme expression in said tumor sample is indicative of a cancer patient who is not a candidate for arginine deprivation therapy. Prior to, simultaneous with, or after testing the tumor sample, the method further comprises the steps of obtaining a non-cancerous sample from the cancer patient and detecting the presence or absence of evidence of urea cycle enzyme expression in the non-cancerous sample, wherein the absence of evidence of urea cycle enzyme expression in the non-cancerous sample and absence of evidence of urea cycle enzyme expression in the tumor sample is indicative of a cancer patient who is not a good candidate for arginine deprivation therapy, the presence of evidence of urea cycle enzyme expression in the non-cancerous sample and the absence of evidence of urea cycle enzyme expression in the tumor sample is indicative of a cancer patient who is a good candidate for arginine deprivation therapy, and the presence of evidence of urea cycle enzyme expression in the tumor sample is indicative of a cancer patient who is not a candidate for arginine deprivation therapy.

### FIELD OF THE INVENTION

The present invention relates generally to the field of oncology. More particularly, the invention relates to methods for treating cancer and methods for predicting the susceptibility of cancer patients to arginine deprivation therapy.

### BACKGROUND OF THE INVENTION

Malignant melanoma (stage 3) and hepatoma are fatal diseases that kill most patients within one year of diagnosis. In the United States, approximately 16,000 people die from these two diseases annually. The incidence of melanoma is increasing rapidly in the United States and is even higher in other countries, such as Australia. Hepatoma is the most common cancer in the world with about one million new cases every year. Roughly 20,000 new cases are diagnosed every year in United States. The incidence of hepatoma in parts of the world where hepatitis is endemic is even greater. For example, hepatoma is one of the most common forms of cancer in Japan and Taiwan.

Sarcoma is a relatively rare but often deadly cancer. Approximately 8,100 Americans will be diagnosed with soft-tissue sarcomas this year, and some 4,600 will die of the disease. Sarcoma is a general class of uncommon cancers in which the cancer cells arise from or resemble normal cells in the body known as “connective tissues”. Normal “connective tissues” include fat, muscle, blood vessels, deep skin tissues, nerves, bones, and cartilage. Sarcomas are sub-classified based upon the specific type of cell that makes up the cancer.

Breast cancer is the most common cancer among women, excluding non-melanoma skin cancers. Breast cancer is the second leading cause of cancer death in women, exceeded only by lung cancer. The American Cancer Society estimates that about 180,000 new cases of invasive breast cancer will be diagnosed among women in the United States in 2000. Breast cancer also occurs in men. An estimated 1,400 cases will be diagnosed annually among men.

There are many different cancer treatment measures in use today, the effectiveness of which varies dramatically. Many of these cancer treatments have undesired side effects including hair loss, compromised immune systems, and nausea. One factor common to all types of cancer and treatments is time of detection and initiation of appropriate treatment. Early detection and initiation of the appropriate treatment improves the patient's prognosis dramatically. There is a longstanding need for methods of treating cancer, especially melanomas, hepatomas, sarcomas, and breast cancers. There is also a need for methods that identify which cancer patients are susceptible to a given treatment.

The selective depletion of amino acids from the circulation by the administration of amino acid degrading enzymes as a treatment for cancer is an idea that has been around for at least 40 years (Muller et al., J. Critical Reviews in Oncology/Hematology 28, 97-113, 1998). The best known example is the use of L-asparaginase to lower circulating levels of asparagine in the treatment if acute lymphoblastic leukemia. Recently it has been reported that arginine-degrading enzymes may prove highly effective in controlling melanoma, hepatoma, and some sarcomas. (Kamisaki et al., Gann. 73, 470-4, 1982; Sugimura et al., Melanoma Res. 2, 191-6, 1992; Takaku et al., Jpn. J. Cancer Res. 86, 840-6, 1995). Human melanomas and hepatomas may be killed by the elimination of arginine from the culture medium, or when implanted into mice, via the injection of arginine deiminanse (ADI; an arginine degrading enzyme) into the host.

In addition, there is reported to be a number of other tumor cells that are killed by ADI. (Takaku et al., Int. J. Cancer 51, 244-9, 1992; Miyazaki et al., Cancer Res. 50, 4522-7, 1990).

However, it is known that arginine deficiency can have undesired side effects on certain patients. Additionally, it has been shown that arginine deprivation therapy is not effective on all tumors.

Therefore, there is a need for methods of determining which cancer patients are susceptible to arginine deprivation therapy. Similarly, there is a need for methods of determining which cancer patients are not susceptible to arginine deprivation therapy and which cancer patients are more susceptible to undesirable side effects. Methods for determining which cancer patients are susceptible to ADI therapy will enable those in the medical community to efficiently initiate the appropriate course of treatment. Patients can receive the appropriate cancer treatment sooner than presently possible avoiding inappropriate or ineffective treatment and saving time and money.

The present invention is directed to these, as well as other, important needs.

### SUMMARY OF THE INVENTION

The present invention addresses the needs identified above in that it provides methods for determining which cancer patients are susceptible to arginine depletion therapy.

In some embodiments, the present invention provides methods comprising obtaining a tumor sample from the cancer patient and detecting the presence or absence of evidence of argininosuccinate synthetase (ASS) expression in the tumor sample. The absence of evidence of ASS expression in the tumor sample is indicative of a cancer patient who is a candidate for arginine deprivation therapy, and the presence of evidence of ASS expression in said tumor sample is indicative of a cancer patient who is not a candidate for arginine deprivation therapy. Prior to, simultaneous with, or after testing the tumor sample, the method further comprises the steps of obtaining a non-cancerous sample from the cancer patient and detecting the presence or absence of evidence of ASS expression in the non-cancerous sample, wherein the absence of evidence of ASS expression in the non-cancerous sample and absence of evidence of ASS expression in the tumor sample is indicative of a cancer patient who is not a good candidate for arginine deprivation therapy, the presence of evidence of ASS expression in the non-cancerous sample and the absence of evidence of ASS expression in the tumor sample is indicative of a cancer patient who is a good candidate for arginine deprivation therapy, and the presence of evidence of ASS expression in the tumor sample is indicative of a cancer patient who is not a candidate for arginine deprivation therapy.

In some embodiments, the present invention provides methods comprising obtaining a tumor sample from the cancer patient and detecting the presence or absence of evidence of argininosuccinate lyase (ASL) expression in the tumor sample. The absence of evidence of ASL expression in the tumor sample is indicative of a cancer patient who is a candidate for arginine deprivation therapy and the presence of evidence of ASL expression in said tumor sample is indicative of a cancer patient who is not a candidate for arginine deprivation therapy. Prior to, simultaneous with, or after testing the tumor sample, the method further comprises the steps of obtaining a non-cancerous sample from the cancer patient and detecting the presence or absence of evidence of ASL expression in the non-cancerous sample, wherein the absence of evidence of ASL expression in the non-cancerous sample and absence of evidence of ASL expression in the tumor sample is indicative of a cancer patient who is not a good candidate for arginine deprivation therapy, the presence of evidence of ASL expression in the non-cancerous sample and the absence of evidence of ASL expression in the tumor sample is indicative of a cancer patient who is a good candidate for arginine deprivation therapy, and the presence of evidence of ASL expression in the tumor sample is indicative of a cancer patient who is not a candidate for As arginine deprivation therapy.

In some embodiments, the present invention provides methods of treating a patient who has cancer. The methods comprise the steps of determining if the cancer patient is a candidate for arginine deprivation therapy as described supra and infra. The cancer patient is treated with arginine deprivation therapy if the patient is a candidate for arginine deprivation therapy. The cancer patient is treated with conventional cancer treatment (e.g. non-ADI therapy) if the cancer patient is not a candidate for arginine deprivation therapy.

These and other aspects of the present invention will be elucidated in the following detailed description of the invention.

### BRIEF DESCRIPTION OF THE DRAWINGS

FIG. 1 depicts an agarose gel of RT-PCR of samples from different human tumor cell lines. Lane 1, molecular weight standards (from top to bottom: 5148 bp, 4973 bp, 4268 bp, 3530 bp, 1352 bp, 1078 bp, 872 bp, 603 bp, 310 bp); lane 2, A764 cells; lane 3, HTB-44 cells: lane 4, CRL 1932 cells; lane 5, CRL 1933 cells; lane 6, HB 8064 cells; lane 7, HB 8065 cells; lane 8, WRL 68 cells; lane 9, HEP3B cells; lane 10, HTB92 cells; lane 11, SKHEP3 cells; lane 12, SKHEP2; lane 13, SKHEP1 cells.

FIGS. 2A-2B depict Northern blots of RNA from different human tumor cell lines probed with different cDNA of different urea cycle enzymes. FIG. 2A depicts a Northern blot probed with labeled ASL cDNA. Lane 1, SK-mel 2 melanoma; lane 2, SK-mel 3 melanoma; lane 3, SK-mel 28 melanoma; lane 4, MeWo lymphoma; lane 5, T47-D breast adenocarcinoma; lane 6, A549 lung carcinoma; lane 7, HB 8065 hepatoma; lane 8, HTB 52 hepatoma. FIG. 2B depicts a Northern blot probed with labeled ASS cDNA. Lane 1, SK-mel 2 melanoma; lane 2, SK-mel 3 melanoma; lane 3, SK-mel 28 melanoma; lane 4, MeWo lymphoma; lane 5, T47-D breast adenocarcinoma; lane 6, A549 lung carcinoma; lane 7, HB 8065 hepatoma; lane 8, HTB 52 hepatoma.

FIG. 3 depicts the effect of ADI on human melanoma cells transfected with ASS.

FIG. 4 depicts a Western blot using an anti-ASS antibody. Lane 1, pre-stained molecular weight markers (top to bottom in KDa: 98, 64, 50, 36, 30, 16, 6); lane 2, purified recombinant ASS.

### DETAILED DESCRIPTION OF THE PREFERRED EMBODIMENTS

Various definitions are made throughout this document. Most words have the meaning that would be attributed to those words by one skilled in the art. Words specifically defined either below or elsewhere in this document have the meaning provided in the context of the present invention as a whole and as are typically understood by those skilled in the art.

As used herein, the phrase “urea cycle enzyme” refers to enzymes involved in the synthesis of arginine from citrulline. Examples of urea cycle enzymes include, but are not limited to ASS and ASL.

As used herein, the term “susceptible” refers to patients for whom arginine deprivation therapy is an acceptable method of treatment, i.e., patients who are likely to respond positively. Cancer patients susceptible to arginine deprivation therapy lack evidence of urea cycle enzyme expression. Cancer patients who are not good candidates for arginine deprivation include cancer patients with tumor samples that do not lack evidence of urea cycle enzyme expression as well as those for whom arginine deprivation therapy would cause undesired side effects, including those patients whose non-cancerous samples lack evidence of urea cycle enzyme expression.

As used herein, the term “sample” refers to biological material from a patient. The sample assayed by the present invention is not limited to any particular type. Samples include, as non-limiting examples, single cells, multiple cells, tissues, tumors, biological fluids, biological molecules, or supernatants or extracts of any of the foregoing. Examples include tissue removed for biopsy, tissue removed during resection, blood, urine, lymph tissue, lymph fluid, cerebrospinal fluid, mucous, and stool samples. The sample used will vary based on the assay format, the detection method and the nature of the tumors, tissues, cells or extracts to be assayed. Methods for preparing samples are well known in the art and can be readily adapted in order to obtain a sample that is compatible with the method utilized. As used herein, the term “biological molecule” includes, but is not limited to, proteins, nucleic acids, and saccharides.

As used herein, the term “detecting” means to establish, discover, or ascertain evidence of urea cycle enzyme expression. Methods of detection are well known to those of skill in the art. For example, methods of detecting urea cycle enzyme polynucleotides include, but are not limited of PCR, Northern blotting, Southern blotting, RNA protection, and DNA hybridization (including in situ hybridization). Methods of detecting urea cycle enzyme polypeptide include, but are not limited to, Western blotting, ELISA, enzyme activity assays, slot blotting, peptide mass fingerprinting, electrophoresis, and immunohistochemistry. Other examples of detection methods include, but are not limited to, radioimmunoassay (RIA), chemiluminescence immunoassay, fluoroimmunoassay, time-resolved fluoroimmunoassay (TR-FIA), or immunochromatographic assay (ICA), all well known by those of skill in the art. For example, in the context of “detecting the presence of ASS” is meant to refer to establishing that evidence of ASS expression is present. In preferred embodiments of the present invention, ASS and/or ASL is detected using ELISA or PCR methodologies.

As used herein, the term “presence” refers to establishing that the item in question is detected in levels greater than background. Presence may, for example, refer to the presence of homology to a given sequence or the presence of binding to a target.

As used herein, the term “absence” refers to establishing that the item in question is not detected in levels greater than background or is undetectable.

As used herein, the phrase “evidence of urea cycle enzyme expression” refers to any measurable indicia that a urea cycle enzyme is expressed in the sample. Evidence of urea cycle enzyme expression may be gained from methods including, but not limited to, PCR, FISH, ELISA, or Western blots.

As used herein, the phrase “arginine deprivation therapy” refers to a treatment regimen that involves the use of an agent that reduces, minimizes, or abolishes arginine levels in the patient. Arginine deprivation therapy is often performed using ADI. Arginine deprivation therapy and agents used in arginine deprivation therapy are described in detail in allowed U.S. application Ser. No. 09/023,809, filed Feb. 13, 1998; and pending application Ser. No. 09/504, 280, filed Feb. 15, 2000, each of which is hereby incorporated by reference in its entirety.

As used herein, the term “melanoma” refers to a malignant or benign tumor arising from the melanocytic system of the skin and other organs, including the oral cavity, esophagus, anal canal, vagina, leptomeninges, and/or the conjunctivae or eye. The term “melanoma” includes, as non-limiting examples, acral-lentiginous melanoma, amelanotic melanoma, benign juvenile melanoma, lentigo maligna melanoma, malign melanoma, nodular melanoma, subungual melanoma, and superficial spreading melanoma.

As used herein, “hepatoma” may be a malignant or benign tumor of the liver, including as non-limiting examples, hepatocellular carcinoma, malignant liver tumor, fibrolamellar hepatoma, and hepatocellular cholangiocarcinoma, mixed hepatocellular cholangiocarcinoma and undifferentiated hepatocellular carcinoma.

As used herein, “sarcoma” refers to a malignant or benign tumor arising in the connective tissue including as non-limiting examples, bones, cartilage, and striated muscle. Examples of sarcomas include, but are not limited to, liposarcomas, leiomyosarcomas, rhabdomyosarcoma, synovial sarcoma, angiosarcoma, fibrosarcoma, neurofibrosarcoma, gastrointestinal stromal tumor (GIST), Ewing's Sarcoma, osteosarcoma, and chondrosarcoma.

As used herein, “breast cancer” refers to a malignant or benign tumor arising in the breast, and includes, but is not limited to local stage breast cancers, regional stage tumors, and distant stage cancers. Examples of breast cancer include, but are not limited to, adenocarcinoma (including ductal carcinomas and lobular carcinomas), lobular carcinoma in situ (LCIS) medullary carcinoma, mucinous carcinoma, Paget's disease of the nipple, Phyllodes tumor, and tubular carcinoma.

As used herein, the term “candidate for arginine deprivation therapy” refers to a patient for whom arginine deprivation therapy may be beneficial. “Good candidates for arginine deprivation therapy” include patients having non-cancerous samples that possess evidence of urea cycle enzyme expression and tumor samples that lack evidence of urea cycle enzyme expression. Other “candidates for arginine deprivation therapy” include patients having tumor samples that lack evidence of urea cycle enzyme expression. Patients who are “not candidates” for arginine deprivation therapy include patients with tumor sample with evidence of urea cycle enzyme expression. Patients who are “not good candidates for arginine deprivation therapy” include those patients that lack evidence of urea cycle enzyme expression in both non-cancerous samples and in tumor samples. Patients who are “not good candidates for arginine deprivation therapy” may, in certain situations, still be considered as candidates for arginine deprivation therapy”.

As used herein, the phrase “conventional cancer treatment” , also referred to as “non-ADI therapy”, refers to methods of treating cancer other than arginine deprivation therapy. Such conventional treatments include, but are not limited to, chemotherapy, radiation therapy, surgery, hormonal therapy, immunotherapy, cytokine therapy, anti-angiogenesis therapy, and vaccine therapy.

As used herein, the terms “patient” and “donor” are used interchangeably and refer to an animal, preferably a mammal, more preferably a human.

As used herein, the term “appropriateness” refers to determining whether a particular patient is susceptible to arginine deprivation therapy. Cancer patients for whom arginine deprivation therapy is appropriate include, but are not limited to, those cancer patients lacking urea cycle enzymes including but not limited to ASS and ASL.

As used herein, the term “determining” refers to the process of selecting patients for whom arginine deprivation therapy would be effective and selecting those patients for whom arginine deprivation therapy would not be effective. For example, cancer patients for whom arginine deprivation therapy would be effective can be “determined”, inter alia, by detecting the presence or absence of evidence of urea cycle enzyme expression. If, for example, urea cycle enzyme expression is not detected in a patient's non-cancerous sample, then it would be “determined” that arginine deprivation therapy would not, in most situations, be the most effective treatment for the cancer patient. Conversely, if urea cycle enzyme expression is not detected in a cancer patient's sample and evidence of urea cycle enzyme expression was detected in a non-cancerous sample, then it would be “determined” that arginine deprivation would be effective for the cancer patient.

“Synthesized” as used herein, refers to polynucleotides produced by purely chemical, as opposed to enzymatic, methods. “Wholly” synthesized DNA sequences are produced entirely by chemical means, and “partially” synthesized DNAs are those wherein only portions of the r resulting DNA were produced by chemical means.

As used herein, the term “biomolecule” refers to, without limitation, proteins, nucleic acids, saccharides and oligosaccharides.

The term “region” refers to a physically contiguous portion of the primary structure of a biomolecule. In the case of proteins, a region is defined by a contiguous portion of the amino acid sequence of that protein.

The term “domain” as used herein refers to a structural part of a biomolecule that contributes to a known or suspected function of the biomolecule. Domains may be co-extensive with regions or portions thereof and may also incorporate a portion of a biomolecule that is distinct from a particular region, in addition to all or part of that region.

As used herein, the term “antibody” refers to monoclonal and polyclonal antibodies, single chain antibodies, chimeric antibodies, bifunctional/bispecific antibodies, humanized antibodies, human antibodies, and complementary determining region (CDR)-grafted antibodies, including compounds which include CDR sequences which specifically recognize the urea cycle enzyme polypeptide) that are specific for the urea cycle enzyme protein or fragments thereof. Preferred antibodies of the invention are human antibodies which can readily be produced and identified by those skilled in the art. Antibody fragments, including Fab, Fab′, F(ab′)₂, and Fv, are also provided by the invention.

The term “specific for,” when used to describe antibodies of the present invention, indicates that the variable regions of the antibodies of the invention recognize and bind urea cycle enzyme polypeptides exclusively (i.e., are able to distinguish urea cycle enzyme polypeptides from other known polypeptides and other known urea cycle enzyme polypeptides by virtue of measurable differences in properties including binding affinity, despite the possible existence of localized sequence identity, homology, or similarity between the urea cycle enzyme protein and other polypeptides). Those skilled in the art readily understood that such specific antibodies may also interact with other proteins (for example, S. aureus protein A or other antibodies in ELISA techniques) through interactions with sequences outside the variable region of the antibodies, and, in particular, in the constant region of the molecule. Screening assays to determine binding specificity of an antibody of the invention are well known and routinely practiced in the art, as discussed in Harlow et al. (Eds.), Antibodies A Laboratory Manual; Cold Spring Harbor Laboratory; Cold Spring Harbor, N.Y. (1988), Chapter 6.

As used herein, the term “binding” means the physical or chemical interaction between two or more biomolecules or compounds or associated biomolecules or compounds or combinations thereof. Binding includes ionic, non-ionic, Hydrogen bonds, Van der Waals, hydrophobic interactions, etc. Binding can be either direct or indirect, indirect being through or due to the effects of another biomolecule or compound. Direct binding refers to interactions that do not take place through or due to the effect of another biomolecule or compound but instead are without other substantial chemical intermediates.

As used herein, the term “complementary” refers to Watson-Crick basepairing between nucleotide units of a nucleic acid molecule.

As used herein, the term “contacting” means bringing together, either directly or indirectly, a polypeptide or polynucleotide into physical proximity to a polypeptide or polynucleotide of the invention. The polypeptide or polynucleotide can be in any number of buffers, salts, solutions, etc. “Contacting” includes, for example, placing a polynucleotide into a beaker, microtiter plate, cell culture flask, or a microarray, or the like, which contains a nucleic acid molecule. Contacting also includes, for example, placing an antibody into a beaker, microtiter plate, cell culture flask, or microarray, or the like, which contains a polypeptide.

As used herein, the phrase “homologous nucleotide sequence,” or “homologous amino acid sequence,” or variations thereof, refers to sequences characterized by a homology, at the nucleotide level or amino acid level, of at least a specified percentage. Homologous nucleotide sequences include those sequences coding for isoforms of proteins. Such isoforms can be expressed in different tissues of the same organism as a result of, for example, alternative splicing of RNA. Alternatively, isoforms can be encoded by different genes. Homologous nucleotide sequences include nucleotide sequences encoding for a protein of a species other than humans, including, but not limited to, mammals. Homologous nucleotide sequences also include, but are not limited to, naturally occurring allelic variations and mutations of the nucleotide sequences set forth herein. Homologous amino acid sequences include those amino acid sequences which contain conservative amino acid substitutions and which polypeptides have the same binding and/or activity.

Percent homology can be determined by, for example, the Gap program (Wisconsin Sequence Analysis Package, Version 8 for Unix, Genetics Computer Group, University Research Park, Madison Wis.), using default settings, which uses the algorithm of Smith and Waterman (Adv. Appl. Math., 1981, 2, 482-489). In some preferred embodiments, homology between the probe and target is between about 50% to about 60%. In some embodiments, homology is between about 60% to about 70%. In preferred embodiments, homology is between about 70% and about 80%. In more preferred embodiments, homology is between about 80% and about 90%. In most preferred embodiments, homology is between about 90% and 100%.

As used herein, the term “isolated” nucleic acid molecule refers to a nucleic acid molecule (DNA or RNA) that has been removed from its native environment. Examples of isolated nucleic acid molecules include, but are not limited to, recombinant DNA molecules contained in a vector, recombinant DNA molecules maintained in a heterologous host cell, partially or substantially purified nucleic acid molecules, and synthetic DNA or RNA molecules.

As used herein, the term “oligonucleotide” refers to a short series of linked nucleotide residues to be used in a polymerase chain reaction (PCR). This short sequence is based on (or designed from) a genomic sequence or cDNA sequence and is used to amplify, confirm, or detect the presence of an identical, similar, or complementary DNA or RNA in a particular cell or tissue. Oligonucleotides comprise portions of a DNA sequence having at least about 10 nucleotides and as many as about 50 nucleotides, preferably from about 15 nucleotides to about 30 nucleotides, and even more preferably from about 20 nucleotides to about 25 nucleotides. Oligonucleotides may be chemically synthesized and can also be used as probes.

As used herein, the term “probe” refers to nucleic acid sequences of variable length, preferably between at least about 10 and as many as about 6,000 nucleotides, depending on the desired use. Preferred probes comprise at least 12, 14, 16, 18, 20, 25, 50, or 75 consecutive nucleotides. Probes are used in the detection of identical, similar, or complementary nucleic acid sequences. Longer length probes are usually obtained from natural or recombinant sources, are highly specific to the target sequence, and are much slower to hybridize to the target than are oligomers. Probes may be single- or double-stranded and are designed to have specificity in PCR, hybridization membrane-based, in situ hybridization (ISH), fluorescent in situ hybridization (FISH), or ELISA-like technologies.

As used herein, the phrase “stringent hybridization conditions” or “stringent conditions” refers to conditions under which a probe, primer, or oligonucleotide will hybridize to its target sequence, but to no other sequences. Specific stringent conditions are sequence-dependent and will be different in different circumstances. Longer sequences generally hybridize specifically at higher temperatures. Typically, stringent conditions are selected to be about 5° C. lower than the thermal melting point (Tₘ) for the specific sequence at a defined ionic strength and pH. The Tₘ is the temperature (at defined ionic strength, pH, and nucleic acid concentration) at which 50% of the probes complementary to the target sequence hybridize to the target sequence at equilibrium. Since the target sequences are generally present in excess, at Tₘ, 50% of the probes are occupied at equilibrium. Typically, stringent conditions will be those in which the salt concentration is less than about 1.0 M sodium ion, typically from about 0.01 to about 1.0 M sodium ion (or other salts) at pH 7.0 to 8.3, and the temperature is at least about 30° C. for short probes, primers, or oligonucleotides (e.g. 10 to 50 nucleotides), and at least about 60° C. for longer probes, primers, or oligonucleotides. Stringent conditions may also be achieved with the addition of destabilizing agents, such as formamide.

For example, typical highly stringent hybridization conditions are as follows: hybridization at 42° C. in a solution comprising 50% formamide, 1% SDS, 1 M NaCl, 10% Dextran sulfate and washing twice for 30 minutes each wash at 60° C. in a wash solution comprising 0.1×SSC and 1% SDS. Those skilled in the art understand that conditions of equivalent stringency can also be achieved through varying temperature and buffer, or salt concentration as described by Ausubel et al. (Protocols in Molecular Biology, John Wiley & Sons (1994), pp. 6.0.3 to 6.4.10). Modifications in hybridization conditions can be empirically determined or precisely calculated based on the length and the percentage of guanosine/cytosine (GC) base pairing of the probe. Hybridization conditions can be calculated as described in, for example, Sambrook et al., (Eds.), Molecular Cloning: A Laboratory Manual, Cold Spring Harbor Laboratory Press: Cold Spring Harbor, N.Y. (1989), pp. 9.47 to 9.51.

Applicants' discovery solves the important problem of determining which patients are susceptible to arginine deprivation therapy and which patients are better suited to other treatments. Applicants have discovered that the absence of urea cycle enzymes in a cancer patient's sample coupled with the presence of evidence of urea cycle enzyme expression in a non-cancerous sample is correlated to the cancer patient's susceptibility to arginine deprivation therapy. Conversely, the presence of urea cycle enzymes in a patient's tumor sample or the absence of urea cycle enzyme expression in a non-cancerous sample indicates that the patient is not generally a good candidate for arginine deprivation therapy for cancer and is better suited to alternative treatments, e.g., non-ADI therapy.

Arginine is not an essential amino acid for most cells because it may be synthesized from citrulline in two steps via the urea cycle enzymes, argininosuccinate synthetase (ASS) and argininosuccinate lyase (ASL) (Brusilow et al, “Urea cycle enzymes,” Chap. 32 in The Metabolic and molecular Basis of Inherited Diseases, 7^{th} Edition, Eds. Scriver, Beauddet, Sly, and Valle, McGraw-Hill, New York, 1995, pp. 1187-1232.) ASS catalyzes the conversion of citrulline and aspartic acid to argininosuccinic acid. Argininosuccinic acid is then converted to arginine and fumaric acid by ASL. Expression of ASS and ASL is more or less ubiquitous (Wakabayashi, Curr. Opin. Clin. Nutr. Metab. Care 1, 335-9, 1998; Yu et al, J. Biochem (Tokyo) 117, 952-7, 1995). Therefore, most normal cells have the ability to convert citrulline into arginine.

ADI converts arginine into citrulline. Citrulline as discussed above, in turn, can be taken up by normal cells and re-converted back into arginine. The uptake mechanism of citrulline is complex and subject to regulation by ions and possibly G proteins. In addition several mutations in citrulline metabolism have been mapped to chromosome 7q21.3 and 9q34.

In normal tissues, a deficiency in urea cycle enzymes results in an undesired accumulation of citrulline in the blood (citrullinemia) (Brusilow, above). Citrullinemia can result on elevated blood ammonia levels and neurological symptoms including coma. Most patients with the severe forms (Types I and III citrullinemia) of this disorder are diagnosed at birth. However, a late onset form (Type II citrullinemia) occurs in adult life and is not frequently detected. Urea cycle enzyme deficiency is uncommon in the United States and Europe, but Type II citrullinemia is most common in Japan where about 1:100,000 persons have the disorder. The disease is inherited in an autosomal recessive manner. The gene is located on chromosome 7 (Sianasac et al., Genomics 62, 289-92, 1999). Screening of patients for type II citrullinemia before ADI therapy may prevent adverse side effects not seen in patients with normal urea cycle enzyme levels. The method comprises obtaining a patient sample and detecting the presence or absence of urea cycle enzyme expression in said sample. The absence of urea cycle enzymes in non-cancerous cells may be indicative of a patient with type II citrullinemia for whom ADI therapy is not appropriate.

Those persons with urea cycle enzyme deficiency in non-cancerous cells who were treated with ADI therapy would be expected to have even more marked elevation of serum citrulline levels. This is due to a failure to convert citrulline to argininosuccinic acid (due to the ASS deficiency), and enhanced arginine conversion to citrulline (due to an ADI therapy). Therefore, ADI treatment in ASS deficient patients would actually result in the undesired elevation of serum citrulline levels.

There are a number of ways a tumor cell may become auxotrophic for arginine, that is, unable to synthesize arginine. For example, there could be a defect in the mechanism for the uptake of citrulline by the tumor cell from the blood. As a result, the tumor cell would not be able to convert citrulline to arginine for use by the cell. It is also possible that tumor cells have a deficiency in the activities or presence of one or more urea cycle enzymes, in particular, ASS or ASL. These enzymes may be completely absent from tumor cells because the genes encoding them are not expressed, therefore not producing any mRNA which could be translated into functioning enzymes. Similarly, the genes may contain mutations which result in defective, non-functioning enzymes, or enzymes with less than optimal activities. Another possibility is that there is a defect in the translational mechanism that converts the specific mRNAs into the enzyme, thereby resulting in the absence of the enzymatic activities in tumor cells.

Although, as discussed above, there are a number of possible different mechanisms that could result in a tumor becoming sensitive to arginine deprivation, the exact mechanism was unknown until the present invention.

The present invention is based on the surprising discovery that one may predict which cancer patients are susceptible to arginine deprivation therapy based on the absence of one or more enzymes involved in arginine synthesis. The present invention provides methods for determining which cancer patients are susceptible to arginine deprivation therapy. Through a series of experiments set out in detail in the following Examples, the inventors determined that the inability to synthesize arginine is due to a deficiency in the urea cycle enzymes.

Surprisingly, it has been found that 100% of human melanomas and human hepatomas tested thus far were ASS negative and sensitive to ADIkilling. Further 60% of human sarcomas were ASS negative and sensitive to ADI killing. Even though a number of breast cancer cell lines tested in vitro were found to be ASS negative by RT-PCR and a single biopsy out of a total of 17 breast cancer samples was found to be negative by immunohistochemical staining, none of these breast tumor cells were tested for sensitivity to ADI killing.

Therefore, by assaying for the absence or presence of evidence of urea cycle enzyme expression in cancer patients' samples, medical practitioners can determine which cancer patients are susceptible to arginine deprivation therapy, and also which cancer patients are not susceptible to arginine deprivation therapy, thereby identifying appropriate treatment regimens.

In one embodiment, the present invention provides methods for identifying cancer patients susceptible to arginine deprivation therapy. The method comprises obtaining a tumor sample from the cancer patient and detecting the presence or absence of evidence of argininosuccinate synthetase (ASS) expression in the tumor sample. The absence of evidence of ASS expression in the tumor sample is indicative of a cancer patient who is a candidate for arginine deprivation therapy, and the presence of evidence of ASS expression in said tumor sample is indicative of a cancer patient who is not a candidate for arginine deprivation therapy. Prior to, simultaneous with, or after testing the tumor sample, the method further comprises the steps of obtaining a non-cancerous sample from the cancer patient and detecting the presence or absence of evidence of ASS expression in the non-cancerous sample, wherein the absence of evidence of ASS expression in the non-cancerous sample and absence of evidence of ASS expression in the tumor sample is indicative of a cancer patient who is not a good candidate for arginine deprivation therapy, the presence of evidence of ASS expression in the non-cancerous sample and the absence of evidence of ASS expression in the tumor sample is indicative of a cancer patient who is a good candidate for arginine deprivation therapy, and the presence of evidence of ASS expression in the tumor sample is indicative of a cancer patient who is not a candidate for arginine deprivation therapy. Evidence of ASS expression may include, but is not limited to, ASS protein or mRNA that encodes ASS.

In another embodiment, the present invention provides methods for identifying cancer patients susceptible to arginine deprivation therapy. The method comprises obtaining a tumor sample from the cancer patient and detecting the presence or absence of evidence of argininosuccinate lyase (ASL) expression in the tumor sample. The absence of evidence of ASL expression in the tumor sample is indicative of a cancer patient who is a candidate for arginine deprivation therapy, and the presence of evidence of ASL expression in said tumor sample is indicative of a cancer patient who is not a candidate for arginine deprivation therapy. Prior to, simultaneous with, or after testing the tumor sample, the method further comprises the steps of obtaining a non-cancerous sample from the cancer patient and detecting the presence or absence of evidence of ASL expression in the non-cancerous sample, wherein the absence of evidence of ASL expression in the non-cancerous sample and absence of evidence of ASL expression in the tumor sample is indicative of a cancer patient who is not a good candidate for arginine deprivation therapy, the presence of evidence of ASL expression in the non-cancerous sample and the absence of evidence of ASL expression in the tumor sample is indicative of a cancer patient who is a good candidate for arginine deprivation therapy, and the presence of evidence of ASL expression in the tumor sample is indicative of a cancer patient who is not a candidate for arginine deprivation therapy. Evidence ASL expression may include, but is not limited to, ASL protein or mRNA that encodes ASL.

In some embodiments, the presence or absence of urea cycle enzyme expression is detected using a technique selected from the group consisting of PCR, Northern blotting, Southern blotting, RNA protection, FISH, and DNA hybridization. In other embodiments, the presence or absence of urea cycle enzyme expression is detected using a technique selected from the group consisting of Western blotting, ELISA, enzyme assays, slot blotting, peptide mass fingerprinting, electrophoresis, and immunohistochemistry. In one preferred embodiment, the presence or absence of evidence of urea cycle enzyme expression is determined using PCR. In another preferred embodiment, the presence or absence of evidence of urea cycle enzyme expression is determined using ELISA.

In some embodiments, the method of the present invention provides detects urea cycle enzyme expression in a sample comprising tumor cells (cancer cells). In some preferred embodiments, the sample comprises hepatoma, melanoma, sarcoma, or breast cancer cells. In other embodiments, the sample comprises non-cancerous cells.

In another embodiment of the present invention, the sample is further processed prior to, simultaneously with, or subsequent to said detection of the presence or absence of evidence of urea cycle enzyme expression in the sample. For example, for use in PCR-type assays, nucleic acids are isolated from the sample using techniques well known to those of skill in the art. In other embodiments, for use in immunohistochemical detection of urea cycle enzyme expression, the sample may be embedded in materials such as paraffin prior to detection of urea cycle enzyme expression. In other embodiments, cells may be permeabilized to ensure access of the antibody to antigen inside the cells using, for example, an organic solvent or nonionic detergent. In still other embodiments, cells are fixed by any number of a wide range of available fixatives well known to those of skill in the art. Fixatives include, but are not limited to, organic solvents, such as methanol, acetone, acidified alcohol solutions, or mixtures of solvents. Protein cross-linking reagents such as paraformaldehyde or glutaraldehyde can also be used to fix cells.

In some embodiments the present invention provides methods of treating a patient with cancer. The method comprises the steps of determining if the cancer patient is a candidate for arginine deprivation therapy as described supra and infra. The cancer patient is treated with arginine deprivation therapy if the patient is a candidate for arginine deprivation therapy. The cancer patient is treated with conventional cancer treatment (e.g. non-ADI therapy) if the cancer patient is not a candidate for arginine deprivation therapy.

In other embodiments, the present invention provides methods of treating a patient with breast cancer. The method comprises the steps of determining if the breast cancer patient is a candidate for arginine deprivation therapy as described supra and infra wherein the tumor sample comprises breast cancer cells. The cancer patient is treated with arginine deprivation therapy if the patient is a candidate for arginine deprivation therapy. The breast cancer patient is treated with conventional breast cancer treatment (e.g. non-ADI therapy) if the breast cancer patient is not a candidate for arginine deprivation therapy.

As used herein, the term “screening” refers to the examination of a sample for evidence of the presence or absence of evidence of urea cycle enzyme expression.

Evidence of ASS expression can be detected comprising the steps of contacting the tumor sample of the cancer patient with a nucleic acid probe which hybridizes under hybridization assay conditions to a nucleic acid target region of a polypeptide having the sequence of SEQ ID NO:7. The probe comprises the nucleic acid sequence encoding the polypeptide, fragments thereof, and the complements of the sequences and fragments. The binding of the nucleic acid probe to the nucleic acid target region is detected. In some other embodiments, the nucleic acid molecule probe has a sequence selected from the group consisting of SEQ ID NO:3 and SEQ ID NO:4.

Evidence of ASL expression can be detected comprising the steps of contacting the tumor sample of the cancer patient with a nucleic acid probe which hybridizes under hybridization assay conditions to a nucleic acid target region of a polypeptide having the sequence of SEQ ID NO:8. The probe comprises the nucleic acid sequence encoding the polypeptide, fragments thereof, and the complements of the sequences and fragments. The binding of the nucleic acid probe to the nucleic acid target region is detected. In some other embodiments, the nucleic acid molecule probe has a sequence selected from the group consisting of SEQ ID NO:9 and SEQ ID NO:10.

In some embodiments, the nucleic acid probe has a detectable label. In some preferred embodiments, the detectable label is radioactive, fluorescent, or chromomorphic. In some more preferred embodiments, the detectable label is ¹³¹I, ¹²⁵I, ¹⁴C, ³⁵S, ³²P, or ³³P.

In other embodiments, the detectable label is fluorescein, phycolipoprotein, or tetrarhodamine isothiocyanate.

In other embodiments, the detectable label is an enzyme. Examples of enzymes suitable for detection include, but are not limited to, alkaline phosphatase, horseradish peroxidase, and luciferase.

Evidence of ASS expression can also be detected comprising the steps of contacting the tumor sample of the cancer patient with at least one ASS-specific polynucleotide or complement thereof. Binding of the ASS-specific polynucleotide or complement thereof to a target in said tumor sample is detected.

Evidence of ASL expression can also be detected comprising the steps of contacting the tumor sample of the cancer patient with at least one ASL-specific polynucleotide or complement thereof. Binding of the ASL-specific polynucleotide or complement thereof to a target in said tumor sample is detected.

Evidence of ASS expression can also be detected comprising the steps of amplifying a tumor nucleic acid sample of a cancer patient with at least one nucleic acid molecule primer having at least a portion of a nucleotide sequence of SEQ ID NO:1. A determination is made whether a product of the amplification is homologous to the sequence of SEQ ID NO:1, or portion thereof.

Evidence of ASL expression can also be detected comprising the steps of amplifying a tumor nucleic acid sample of a cancer patient with at least one nucleic acid molecule primer having at least a portion of a nucleotide sequence of SEQ ID NO:2. A determination is made whether a product of the amplification is homologous to the sequence of SEQ ID NO:2, or portion thereof.

Evidence of ASS expression can also be detected comprising the steps of contacting the tumor sample of the cancer patient with an antibody directed to an ASS protein, or portion thereof.

Evidence of ASL expression, can also be detected comprising the steps of contacting the tumor sample of the cancer patient with an antibody directed to an ASL protein, or portion thereof.

In some embodiments, the antibody is a labeled, monoclonal or polyclonal, intact, Fab, Fab′, or F(ab′)₂ antibody. The antibodies can be labeled directly or can be detected by using a labeled secondary reagent that will bind specifically to the primary antibody. Exemplary secondary reagents include, but are not limited to, anti-immunoglobulin antibodies, protein A or protein G, or, if the primary antibody is labeled with biotin, streptavidin. Secondary reagents can be labeled with, for example, enzymes, fluorochromes, gold, or iodine. Enzymes that can be linked to secondary reagents include, but are not limited to, horseradish peroxidase, alkaline phosphatase, or β-galactosidase. Fluorochromes include, for example, fluorescein, DAPI, and rhodamine. In other embodiments, the detectable label is radioactive. In some more preferred embodiments, the detectable label is ¹³¹I, ¹²⁵I, ¹⁴C, ²⁵S, ³²P, or ³³P.

The antigen used for producing the anti-urea cycle enzyme antibody is not limited to any particular antigen so long as it contains a peptide derived from urea cycle enzymes. The source of the urea cycle enzymes used for the antigen is not limited in terms of its production process. The antigen used for producing the urea cycle enzyme antibody may be one that is derived from a natural organism or produced by genetic engineering means or chemical synthesis, or may be derived in part from a natural organism and in part by genetic engineering or chemical synthesis. Examples of antigens suitable for the present invention include, but are not limited to, the in its full length, a peptide fragment derived from ASS or ASL, a deletion or substitution mutant of ASS or ASL wherein one or more amino acid has been deleted or substituted, and a fusion protein including a part of the ASS or ASL, or fused, naturally non-contiguous portions of ASS or ASL.

Kits comprising an antibody of the invention are also contemplated. In general, a kit optionally also includes a control antigen for which the antibody is immunospecific, instructions for use, positive and negative controls, molecular weight markers, illustrations of exemplary results, and containers.

Kits comprising one or more polynucleotides of the invention are also contemplated. In general, a kit optionally also includes a control sequence to which the probe can hybridize (positive control), a control sequence to which the probe does not hybridize (negative control) instructions for use, molecular weight markers, illustrations of exemplary results, and containers.

Another aspect of the present invention is the use of the urea cycle enzyme nucleotide sequences disclosed herein for identifying homologs of the ASS or ASL nucleotide sequences, in other organisms. Any of the nucleotide sequences disclosed herein, or any portion thereof, can be used, for example, as a probe to screen nucleic acid libraries, such as, for example, genomic or cDNA libraries, to identify homologs, using screening procedures well known to those skilled in the art. Accordingly, homologs having at least 50%, more preferably at least 60%, more preferably at least 70%, more preferably at least 80%, more preferably at least 90%, more preferably at least 95%, and most preferably 100% homology with ASS or ASL nucleotide sequences can be identified.

Nucleotide and amino acid sequences of ASS and ASL are set forth in the attached Sequence Listing, which is incorporated by reference in its entirety. Amino acid sequences represented therein are set forth in the amino to carboxy direction, from left to right. The amino and carboxy groups are not represented in the amino acid sequences. Nucleotide sequences are represented by single strand only, in the 5′ to 3′ direction, from left to right.

The present invention also contemplates embodiments wherein a patient lacking evidence of urea cycle enzyme expression in a tumor sample is treated with arginine deprivation therapy even though the cancer patient's non-cancerous sample lacks evidence of urea cycle enzyme expression. In these embodiments, although the patient may not be a good candidate for arginine deprivation therapy, it may be determined that the need for effective treatment of one or more tumors outweighs risks of possible side-effects associated with the use of arginine deprivation therapy for patients lacking urea cycle enzymes in non-cancerous cells.

The present invention is further demonstrated in the following examples that are for purposes of illustration and are not intended to limit the scope of the present invention. Examples 1-6 are actual, while Example 7 is prophetic.

### EXAMPLES

The manipulations used in the Examples as described below have been performed as generally described by Sambrook et al. ed., “Molecular Cloning, a Laboratory Manual, 2nd ed.”, Cold Spring Harbor Laboratory, 1989; and Harlow and Lane, Antibodies. A Laboratory Manual, Cold Spring Harbor Laboratory, 1988.

### Example 1

### ASS Deficient Cells are Susceptible to Growth Inhibition by ADI Production of ADI

ADI was produced and purified by cloning the gene from Mycoplasma hominus and expressing the protein in E. coli. The gene for M. hominis ADI was isolated using the polymerase chain reaction (PCR). For expression of ADI in E. coli, the expression vector pQE70 (Qiagen) was used. ADI was purified to apparent homogeneity using ion-exchange chromatography. The specific activity of the purified ADI was 20 IU/mg of protein.

Cells and Cell Culture

Cells were obtained from the American Type Culture Collection (“ATCC”; Bethesda, MD), and are listed in Table 1. Sensitivity to ADI was determined by plating the tumor cells in 96 well plates in a volume of 0.1 ml /well. Various concentrations of ADI were added to each well. The plates were incubated for 72 hours at 37° C., then 0.02 ml of alamar blue was added to each well and the plates incubated an additional 5 hours. The absorbance of the wells at 570 nm was then determined using a spectrophotometer. Living cells are able to reduce the dye and the wells become clear, whereas dead cells cannot reduce the dye and the wells remain dark blue in color. Therefore, optical density is inversely proportional to the number of viable cells in culture.

All tested human melanomas and hepatomas were sensitive to ADI killing. About 60% of the tested human sarcomas were sensitive to ADI killing. In contrast, other tumors cell lines tested were able to grow even in the presence of 10 μg/ml of ADI (the highest concentration tested). Table 2 shows the results of these experiments and shows the correlation between sensitivity to ADI and the presence of argininosuccinate synthetase (ASS in these cells. These results are consistent with previously published data (Kamisaki et. al., Gann., 73, 47-474, 1982; Sugimura et. al., Melanoma Res. 2, 191-196 1992; Takaku, et. al., Jpn. J. Cancer Res. 86, 840-846, 1995) and suggest that ADI may be a useful treatment for patients with melanoma, hepatoma, or sarcoma. It is evident from these experiments that not all tumors will be sensitive to ADI treatment. Thus a method of predicting whether or not a tumor is sensitive to ADI before treating a patient would be extremely useful.

TABLE 1
Tumor Cells Tested for Ability to Grow in the Presence of ADI

| Colon | Breast | Liver | Melanoma | Brain | Bladder | Kidney | Lung | Lymphoma |
|---|---|---|---|---|---|---|---|---|
| HT29 | T47D | HB8065 | SKLMEL2 | CCL127 | HTB9 | CRL1933 | A549 | MeWo |
| COLO | BT20 | HB8064 | SKMEL3 | HTB138 | J82 | CRL1932 | | |
| 320HSR | BT74 | CRL8024 | SKMEL37 | HTB148 | T24 | HTB44 | | |
| DLD1 | BT483 | CRL2238 | SKMEL187 | HTB10 | | A704 | | |
| HCT15 | BT549 | CRL2235 | SKMEL28 | CRL1718 | | CRL1611 | | |
| HCT116 | DU4475 | HTB52 | A375 | HTB12 | | | | |
| LOVO | HBL100 | CRL2234 | HTB67 | HTB13 | | | | |
| LS123 | HS578 | CRL2237 | HTB68 | HTB14 | | | | |
| LS174T | MCF7 | SKHEP1 | HTB70 | HTB11 | | | | |
| LS180 | MDA134 | SKHEP2 | HTB71 | A172 | | | | |
| NCIH548 | T47 | SKHEP3 | CRL1675 | H4 | | | | |
| SKCO1 | ZR751 | HEP3B | CRL1676 | HS683 | | | | |
| SW48 | | CCL13 | C32 | HTB16 | | | | |
| SW403 | | HTB92 | C32TG | HTB17 | | | | |
| SW480 | | HEPG2 | G361 | A172 | | | | |
| SW620 | | WRL68 | HMCB | T98G | | | | |
| SW948 | | | HS294T | HTB15 | | | | |
| SW1116 | | | HS695T | | | | | |
| SW1417 | | | HT144 | | | | | |
| T84 | | | HTB64 | | | | | |
| CCD840 | | | HTB66 | | | | | |
| COTR | | | SKMEL5 | | | | | |
| CCL218 | | | SKMEL24 | | | | | |

TABLE 2
Presence or Absence of Evidence of ASS Expression in Cell Lines

| Tumor Type Present | Cell Line | IC50 (μg/ml) | ASS |
|---|---|---|---|
| Melanoma | SK-mel 2 | <0.01 | Neg |
| | SK-mel 3 | <0.01 | Neg |
| | SK-mel 28 | 0.01 | Neg |
| | SK-mel 37 | 0.10 | Neg |
| | A375 | 0.10 | Neg |
| | HTB67 | <0.01 | Neg |
| | HTB68 | <0.01 | Neg |
| | CRL1675 | 0.01 | Neg |
| | CRL1676 | 0.30 | Neg |
| Liver | SKHEP 1 | <0.01 | Neg |
| | SKHEP 2 | <0.01 | Neg |
| | SKHEP 3 | <0.01 | Neg |
| Breast | T47D | >100 | Pos |
| | BT20 | >100 | Pos |
| Colon | HT29 | >100 | Pos |
| | SW48 | >100 | Pos |
| Lung | A549 | >100 | Pos |
| Lymphoma | MeWo | >100 | Pos |

### Example 2

### Northern Blotting

Experiments were performed to determine the mutation that renders tumors sensitive to ADI treatment. To distinguish which mutation in citrulline metabolism was responsible for tumors becoming sensitive to ADI treatment, Northern blots were performed on mRNA isolated from a large number of tumors. These blots were probed with cDNA encoding ASS (SEQ ID NO:1) or ASL (SEQ ID NO:2).

RNA was isolated from human tumor cell lines grown in culture using guanidine isothiocynate. Approximately 1×10⁸ cells were harvested by centrifugation at 300×g for 5 minutes at 4° C. and then resuspended in 4 M guanidine isothiocyanate containing 2-mercaptoethanol. The cells were homogenized using a Brinkman Polytron™ set on high for 15-30 seconds. One-tenth volume of 2M sodium acetate, pH 4.0, was added to the homogenate and mixed thoroughly. The homogenate was extracted using an equal volume of phenol:chloroform:isoamyl alcohol (25:24:1), and then centrifuged at 10,000×g for 20 minutes at 4° C. The upper, aqueous layer was transferred to a clean tube and the RNA precipitated with an equal volume of isopropanol. The RNA was pelleted by centrifugation at 10,000×g for 15 minutes at 4° C. The pellet was washed with 75% cold ethanol and centrifuged at 10,000×g for 15 minutes at 4° C. The pellet was dried and then resuspended in nuclease-free water.

Approximately 10 μg of RNA from each cell line was then separated by electrophoresis on a formaldehyde/agarose gel. After electrophoresis was complete, the gel was rinsed several times in DEPC-treated water to remove the formaldehyde. The RNA in the gel was transferred to a nitrocellulose membrane [Millipore, Bedford, Mass.] and the membrane baked at 80° C. for 2 hours. Membranes were probed with ³²P labeled ASS cDNA or ³²P labeled ASL cDNA containing the full coding sequences of these genes. The probes were labeled with gamma-³²P-ATP using nick translation. Membranes were prehybridized in prehybridization solution (50% deionized formamide, 5×SSPE, 2× Denhardt's Reagent, 0.1% SDS) at 42° C. for 2 hours. Labeled probe (specific activity approximately 10⁸ cpm/μg) was then added and the hybridization carried out at 42° C. overnight. After hybridization, the membrane was washed twice with 0.1×SCC, 0.1% SDS at 68° C. for 5 minutes each wash. The membrane was then washed in 2×SSC for 10 minutes at room temperature and then exposed to X-ray film [Kodak X-Omat AR film, Fisher Scientific, Pittsburgh, Pa.], overnight at −80° C.

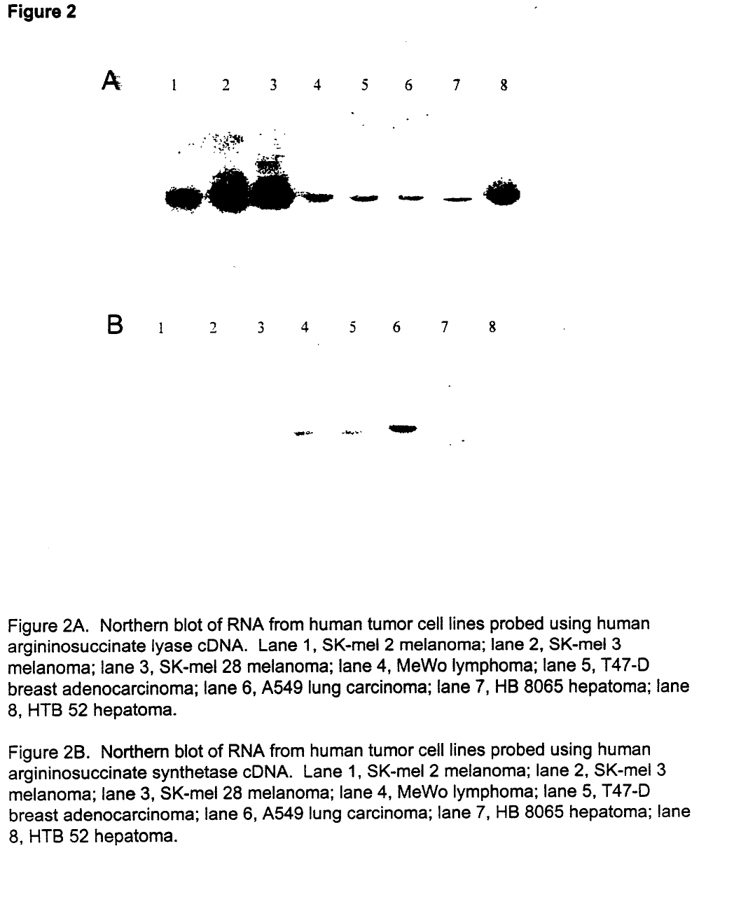

RNA from three different melanomas (SK-mel 2, SK-mel 3, and SK-mel 28), two different hepatomas (HB 8065, and HTB 52), a breast adenocarcinoma (T47-D), a lymphoma (MeWo), and a lung carcinoma (A549) were examined for the presence of ASS and ASL mRNA by Northern blot analysis. The results from the Northern blots clearly demonstrate that melanomas and hepatomas express ASL but fail to express detectable levels of ASS (FIGS. 2A and 2B). The breast adenocarcinoma, lymphoma, and lung carcinoma express both ASL and ASS messages. Only the melanoma and hepatoma cell lines were sensitive to killing by ADI whereas the breast adenocarcinoma, lymphoma, and lung carcinoma cell lines tested were all resistant to ADI.

We observed that ADI sensitive tumors were all deficient in mRNA encoding ASS. Thus the defect was not due to mutations in either chromosome 7q21.3 or 9q34 as these mutations do not suppress ASS mRNA production. Furthermore all tumor cells tested appeared to express ASL. All of the tested tumors that were sensitive to ADI treatment were defective in the ability to produce ASS mRNA. These results demonstrate the utility of using Northern blotting techniques for determining the absence of ASS mRNA in tumor samples and for predicting whether or not a tumor is sensitive to ADI.

### Example 3

### Transfection of Human Melanoma Cells to Constitutively Express the ASS Gene

To prove that the defect in the ADI sensitive cells was due to an inability to express ASS mRNA, ADI sensitive cells which, as shown above in Table 2, do not express ASS, were transfected with an expression plasmid containing the human ASS gene. The human melanoma cell lines SK-mel 2 and SK-mel 28 were transfected by electroporation with the expression plasmid containing the human ASS gene. The plasmid was constructed with the human cDNA encoding ASS under the regulation of a cytomegalovirus promoter. The plasmid also contained a neomycin resistance gene that allowed for selection of the melanoma cells that had been transfected. One million cells of each type were mixed with 50 μg of the expression plasmid and electroporated. Next the cells were plated out in 100 mm petri dishes containing growth medium. After 24 hours G418 was added to the culture to kill cells which had not taken up the expression plasmid. After an additional 3 weeks of growth, isolated clones of the transfected cells were isolated and tested for the ability to grow in ADI.

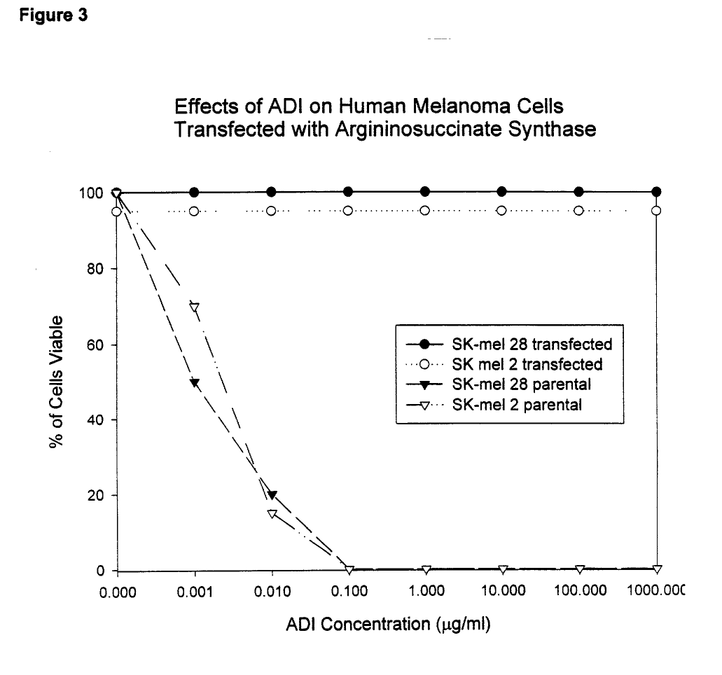

Cells transfected with the ASS gene were challenged with ADI and found to be more than 1000 times more resistant to ADI killing than control cells (untransfected cells) (FIG. 3), demonstrating that absence of ASS activity in tumor cells makes them sensitive to ADI treatment. It also demonstrates that the tumor cells have the ability to transcribe and translate ASS mRNA, if it were normally present, and make ASS protein.

### Example 4

### ASS RT-PCR

ASS polynucleotides can be detected using reverse transcriptase-polymerase chain reaction (RT-PCR). RNA is extracted (as described above) from tumor tissues or cells, or normal tissues or cells, and a cDNA copy is made using reverse transcriptase. Total RNA or purified mRNA can be used in RT-PCR for the production of a cDNA template for the PCR. The cDNA is then used as a template in PCR using primers specific for ASS. If ASS mRNA is present in the sample, a PCR product corresponding in size to the ASS sequence amplified by the specific primers in the PCR can be detected by agarose gel electrophoresis.

The sequences of the primers used for the RT-PCR of ASS are as follows:

| | |
|---|---|
| Forward primer: | |
| 5′-ATGTCCAGCAAAGGCTCCGTG-3′ | (SED ID NO:3) |
| Reverse primer: | |
| 5′-CCGTGTTGCTTTGCGTACTCC-3′ | (SED ID NO:4) |

Those of skill in the art recognize that it is possible generate and use primer pairs based on the nucleotide sequence of ASS other than those listed above using the nucleotide sequence of the ASS gene provided as SEQ ID NO:1.

RNA was isolated from human tumor cell lines grown in culture, using guanidine isothiocyanate. Cells were harvested by centrifugation at 300×g for 5 minutes at 4° C. and then resuspended in 4M guanidine isothiocyanate containing 2-mercaptoethanol. The cells were homogenized using a Brinkman Polytron™ set on high for 15-30 seconds. One-tenth volume of 2M sodium acetate, pH 4.0, was added to the homogenate and mixed thoroughly. The homogenate was extracted using an equal volume of phenol:chloroform:isoamyl alcohol (25:24:1) and then centrifuged at 10,000×g for 20 minutes at 4° C. The upper aqueous layer was transferred to a clean tube and the RNA precipitated with an equal volume of isopropanol. The RNA was pelleted by centrifugation at 10,000×g for 15 minutes at 4° C. The pellet was washed with 75% cold ethanol and centrifuged at 10,000×g for 15 minutes at 4° C. The pellet was dried and then resuspended in nuclease-free water.

Primers were added to approximately 1 μg of total RNA and the mixture was incubated at 94° C. for 2 minutes. The tube was then cooled to 48° C. and AMV reverse transcriptase (Boehringer Mannheim Corporation, Indianapolis, Ind.) was added. First strand cDNA synthesis was carried out by incubation at 48° C. for 60 minutes. Taq DNA polymerase (Perkin Elmer) was then added to the reaction and the reaction incubated at 94° C. for 2 minutes. PCR was then carried out using 40 cycles of 94° C. for 30 seconds, 60° C. for 1 minute, and 72° C. for 1 minute. The PCR products were then analyzed by agarose gel electrophoresis.

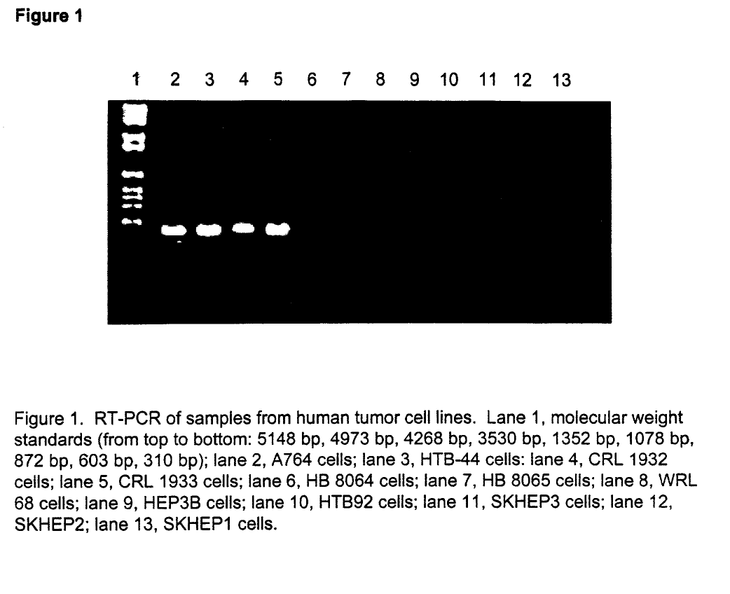

Representative results of RT-PCR analyzed by agarose gel electrophoresis are shown in FIG. 1 which shows results using RNA from a number of different human kidney tumor cell lines, and human hepatocarcinoma cell lines. All the human kidney tumor cells tested contained RNA for ASS and were resistant to killing by ADI. None of the human hepatocarcinoma cells tested contained detectable levels of ASS RNA and were sensitive to killing by ADI.

Table 3 lists the results of RT-PCR performed on a variety of human tumor cell lines, including bladder, breast, colon, kidney, and liver.

TABLE 3

| Tumor Cell Line | Results of RT-PCR |
|---|---|
| Liver | |
| HB8065 | neg |
| HB8064 | neg |
| CRL8024 | neg |
| CRL2238 | neg |
| HTB52 | neg |
| CRL2234 | neg |
| WRL68 | neg |
| HTB92 | neg |
| HEP3B | neg |
| SKHEP1 | neg |
| SKHEP2 | neg |
| SKHEP3 | neg |
| Colon | |
| CCL218 | pos |
| CCD840 | pos |
| T84 | pos |
| SW1417 | pos |
| SW1116 | pos |
| SW948 | pos |
| SW620 | pos |
| SW480 | pos |
| SW403 | pos |
| COLO | pos |
| HT29 | pos |
| 320HSR | pos |
| HCT15 | pos |
| HCT116 | pos |
| LOVO | pos |
| LS123 | pos |
| LS174T | pos |
| 4S180 | pos |
| NCIH548 | pos |
| SKCO1 | pos |
| SW48 | pos |
| SW403 | pos |
| SW480 | pos |
| Breast | |
| BT20 | pos |
| BT74 | neg |
| DU4475 | pos |
| BT549 | neg |
| HBL100 | pos |
| T47D | pos |
| ZR751 | neg |
| HBL100 | pos |
| MDA134 | neg |
| DU4475 | pos |
| BT549 | neg |
| HT144 | pos |
| BT547 | neg |
| Bladder | |
| HTB9 | neg |
| T24 | neg |
| J82 | pos |
| Kidney | |
| A704 | pos |
| HTB44 | pos |
| CRL1933 | pos |
| CRL1933 | pos |

### Example 5

### Preparation of Argininosuccinate Synthetase Antigen

The coding sequence for human ASS was obtained from the American Type Culture Collection (ATCC 57074) as a cDNA insert in pBR322. This plasmid, pAS 4/1/9, was obtained from the ATCC in E. coli HB101.

PCR was used to amplify the ASS gene from pAS 4/1/9 and to place NdeI and NotI restriction endonuclease sites at the 5′ and 3′ ends of the ASS coding sequence, respectively. The PCR primers had the following sequences:

| | |
|---|---|
| ARGSS forNd | |
| 5′CTCCATATGTCCAGCAAAGGCTCCGTG3′ | (SEQ ID NO:5) |
| ARGSS revNo | |
| 5′GAGGCGGCCGCTTTGGCAGTGACCTTG3′ | (SEQ ID NO:6) |

The forward and reverse primers were used in PCR with pAS 4/1/9 as a template and Vent polymerase (New England Biolabs). The PCR reaction used the following conditions: 94° C. for 30 seconds, 50° C. for 30 seconds, 72° C. for 90 seconds for 30 cycles. After the PCR was complete, 1 U of Taq polymerase (Perkin Elmer) was added and the reaction mixture was incubated at 72° C. for 10 minutes. The PCR was run on an agarose gel and the 1256 bp product was excised from the gel and subcloned into pCR2.1 (Invitrogen) to create the plasmid, pCR2.1:ASS. pCR2.1 ASS was digested with NdeI and NotI, and the ASS fragment was subcloned into the NdeI-NotI sites of pET-22b(+) (Novagen). The ligation reaction was used to transform E. coli BL21(DE3), and a transformant expressing ASS protein was isolated. ASS was produced in E. coli as a cytosolic protein. The recombinant protein was purified to homogeneity by chromatography.

### Example 6

### Preparation of Anti-Argininosuccinate Synthetase Antibody

Antibodies to the purified recombinant ASS protein were prepared in rabbits.

Immunizations, test bleedings and final bleeds were performed by Covance, Inc., Princeton, N.J.

Evaluation of Antiserum

An increase in antibody titer against the ASS peptide administered was confirmed by measuring the reactivity between immobilized ASS and the antiserum. Purified ASS was diluted to a final concentration of 1 μg/ml with TBS (100 mM Tris-HCl, 150 mM sodium chloride, pH 8.0). One hundred μL of the diluted ASS was pipetted into each well of a 96 well microtiter plate and incubated overnight at 2 to 8° C. to allow the protein to bind to the wells. The microtiter plate was then rinsed 3 times with TBST (TBS containing 0.5% Tween 20) and 200 μl of TBS containing 5% non-fat dry milk was then added to each well. The plates were incubated at room temperature for 2 hours and then washed 3 times with TBST. Serial dilutions of antiserum were made using TBST containing 5% non-fat dry milk and was then added to the wells. The plate was incubated at room temperature for 1 hour. The wells were then washed 3 times with TBST. Goat-anti-rabbit IgG-alkaline phosphatase conjugate [Jackson ImmunoResearch Laboratories, Inc. West Grove, Pa.], diluted 1:5000 in TBS containing 5% non-fat dry milk, was added to each well and incubated for 30 minutes at room temperature. The wells were then washed 3 times with TBST and then 3 times with TBS. Para-nitrophenyl phosphate in 100 μl alkaline phosphatase buffer was added to each well and the plate was then incubated at room temperature. The optical density of each well was measured at 405nm using a spectrophotometric plate reader.

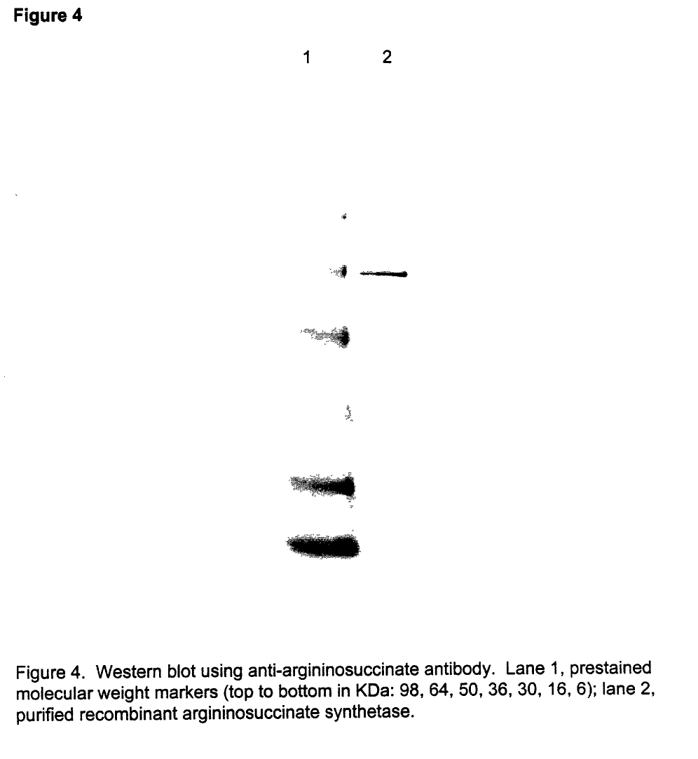

Western blotting was carried out to confirm the binding of the antiserum to the recombinant human ASS. Recombinant human ASS protein was mixed with an equal volume of gel loading buffer, and incubated at 100° C. for 5 minutes. The samples were applied to the wells of a 10 to 20% SDS-PAGE gel (Novex; Invitrogen, Carlsbad, Calif.) and the electrophoresis was performed at 200 V at room temperature until the dye in the sample buffer reached the bottom of the gel. After the completion of the electrophoresis, the protein was transferred from the gel to a PDVF membrane (Millipore) by electrophoresis at 25 mA for 90 minutes. The membrane was blocked by immersion in TBST containing 10% non-fat dry milk for 30 minutes at room temperature with shaking. The membrane was then immersed in 10 ml of the antiserum diluted 1:500 in TBS containing 10% non-fat dry milk, and shaken at room temperature for 1 hour. After the completion of the reaction, the membrane was washed three times with 10 to 20 ml of TBS, and then immersed in 10 ml of anti-rabbit IgG-alkaline phosphatase conjugate (Jackson Laboratories) diluted to 1:5000 in TBS containing 10% non-fat dry milk, and incubated at room temperature for 1 hour. The membrane was then washed 3 times with TBS, and incubated in 10 ml alkaline phosphatase buffer (100 mM Tris/HCl, pH 9.5, 100 mM sodium chloride, 5 mM magnesium chloride) containing 1 mg nitroblue tetrazolium and 2 mg bromo-chloro-indoyl phosphate, until the desired color developed. The membrane was then washed thoroughly with water and allowed to air dry. FIG. 4 shows an example of a Western blot using the anti-ASS antibody.

Purification of the Antiserum

The antiserum was purified by salting out and application through an ion exchange column. To each volume of antiserum an equal volume of saturated ammonium sulfate solution was added slowly with stirring. The stirring was continued at 4° C. for 4 to 6 hours. The mixture was then centrifuged at 10,000×g for 20 minutes, and the resulting precipitate was resuspended in a 1:1 solution of 1×TBS, pH 8.0 (100 mM Tris/HCl, 150 mM sodium chloride)/saturated ammonium sulfate solution equal to the original volume of the antiserum. The solution was then centrifuged at 8,000×g for 20 minutes. The pellet was resuspended in a volume of 1×TBS equal to the original starting volume of the antiserum. Purified argininosuccinate synthetase was coupled to a cyanogen bromide matrix to produce a column that could be used for affinity purification of the anti-ASS antibody. The prepared ASS affinity column was equilibrated with 10 mM Tris/HCl, pH 7.5, and the antibody solution passed through the column 3 times. The column was then washed with 10 mM Tris/HCl, pH 7.5 and then with 10 mM Tris/HCl, pH 7.5 containing 500 mM sodium chloride. The bound antibody was eluted from the column with 50 mM sodium acetate, pH 3.1. Eluted antibody was collected in tubes containing 1M Tris/HCl, pH 8.0. The protein concentrations of purified antibodies were calculated by absorption at 280 nm.

### Example 7

### ASL RT-PCR

ASL polynucleotides can be detected using reverse transcriptase-polymerase chain reaction (RT-PCR). RNA is extracted (as described above) from tumor tissues or cells, or normal tissues or cells, and a cDNA copy is made using reverse transcriptase. Total RNA or purified mRNA can be used in RT-PCR for the production of a cDNA template for the PCR. The cDNA is then used as a template in PCR using primers specific for ASL. If ASL mRNA is present in the sample, a PCR product corresponding in size to the

ASL sequence amplified by the specific primers in the PCR can be detected by agarose gel electrophoresis.

The sequences of the primers used for the RT-PCR of ASL are as follows:

| | |
|---|---|
| Forward primer: | |
| 5′-ATGGCCTCGGAGAGTGGGAAGC-3′ | (SED ID NO:9) |
| Reverse primer: | |
| 5′-TGA CCA CCT GGT CAT TCC GGC TC-3′ | (SED ID NO:10) |

Those of skill in the art recognize that it is possible generate and use primer pairs based on the nucleotide sequence of ASL other than those listed above using the nucleotide sequence of the ASL gene provided as SEQ ID NO:2.

RNA is isolated from human tumor cell lines grown in culture, using guanidine isothiocyanate. Cells are harvested by centrifugation at 300×g for 5 minutes at 4° C. and then resuspended in 4M guanidine isothiocyanate containing 2-mercaptoethanol. The cells are homogenized using a Brinkman Polytron™ set on high for 15-30 seconds. One-tenth volume of 2M sodium acetate, pH 4.0, is added to the homogenate and mixed thoroughly. The homogenate is extracted using an equal volume of phenol:chloroform:isoamyl alcohol (25:24:1) and then centrifuged at 10,000×g for 20 minutes at 4° C. The upper aqueous layer is transferred to a clean tube and the RNA precipitated with an equal volume of isopropanol. RNA is pelleted by centrifugation at 10,000×g for 15 minutes at 4° C. The pellet is washed with 75% cold ethanol and centrifuged at 10,000×g for 15 minutes at 4° C. The pellet is dried and then resuspended in nuclease-free water.

Primers are added to approximately 1 μg of total RNA and the mixture is incubated at 94° C. for 2 minutes. The tube is then cooled to 48° C. and AMV reverse transcriptase (Boehringer Mannheim Corporation, Indianapolis, Ind.) is added. First strand cDNA synthesis is carried out by incubation at 48° C. for 60 minutes. Taq DNA polymerase (Perkin Elmer) is then added to the reaction and the reaction incubated at 94° C. for 2 minutes. PCR is then carried using 40 cycles of 94° C. for 30 seconds, 60° C. for 1 minute, and 72° C. for 1 minute. The PCR products are then analyzed by agarose gel electrophoresis.

Each of the patents, patent applications, and publications described herein is hereby incorporated by reference in its entirety.

Various modifications of the invention, in addition to those described herein, will be apparent to those of skill in the art in view of the foregoing description. Such modifications are also intended to fall within the scope of the appended claims.

1. A method for identifying a cancer patient susceptible to arginine deprivation therapy comprising the steps:
a) obtaining a cancerous tumor sample from the cancer patient; and
b) detecting the presence or absence of argininosuccinate synthetase protein in said cancerous tumor sample, wherein the absence of argininosuccinate synthetase protein in said cancerous tumor sample is indicative of a cancer patient who is a candidate for arginine deprivation therapy and the presence of argininosuccinate synthetase protein in said cancerous tumor sample is indicative of a cancer patient who is not a candidate for arginine deprivation therapy.

2. The method of claim 1 wherein prior to, simultaneous with, or after testing the cancerous tumor sample, the method further comprises the steps of:
c) obtaining a non-cancerous sample of the corresponding tissue from the cancer patient; and
d) detecting the presence or absence of argininosuccinate synthetase protein in said non-cancerous sample, wherein the absence of argininosuccinate synthetase protein in said non-cancerous sample and the absence of argininosuccinate synthetase protein in said cancerous tumor sample is indicative of a cancer patient who is not a good candidate for arginine deprivation therapy, wherein the presence of argininosuccinate synthetase protein in said non-cancerous sample and the absence of argininosuccinate synthetase protein in said cancerous tumor sample is indicative of a cancer patient who is a good candidate for arginine deprivation therapy, and wherein the presence of argininosuccinate synthetase protein in said cancerous tumor sample is indicative of a cancer patient who is not a candidate for arginine deprivation therapy.

3-5. (Canceled)

6. The method of claim 1 wherein the presence or absence of argininosuccinate synthetase protein is detected using a technique selected from the group consisting of Western blotting, ELISA, enzyme assays, slot blotting, electrophoresis, and immunohistochemistry.

7. The method of claim 1 wherein the presence or absence of argininosuccinate synthetase protein is detected using ELISA.

8-26. (Canceled)

27. The method of claim 1 wherein argininosuccinate synthetase protein in said cancerous tumor sample is detected comprising the steps of:
a) contacting the cancerous tumor sample of the cancer patient with an antibody specific for an argininosuccinate synthetase protein, or portion thereof; and
b) detecting binding of the antibody to said argininosuccinate synthetase protein, or portion thereof, in said cancerous tumor sample wherein the absence of binding of the antibody to said argininosuccinate synthetase protein is indicative of a cancer patient who is a candidate for arginine deprivation therapy and the presence of binding of the antibody to said argininosuccinate synthetase protein in said cancerous tumor sample is indicative of a cancer patient who is not a candidate for arginine deprivation therapy.

28-30. (Canceled)

31. The method of claim 27 wherein said antibody has a detectable label.

32. The method of claim 31 wherein said detectable label is radioactive, fluorescent, or chromomorphic.

33. The method of claim 31 wherein said detectable label is ¹³¹I, ¹²⁵I, ¹⁴C, ³⁵S, ³²P, or ³³P.

34. The method of claim 31 wherein said detectable label is fluorescein, phycolipoprotein, or tetrarhodamine isothiocyanate.

35. The method of claim 31 wherein said detectable label is an enzyme.

36. The method of claim 31 wherein said detectable label has a visible color.


SEQUENCE LISTING (10 sequences)

SEQ ID NO: 1 - DNA, 1239 bp, Homo sapiens
atgtccagcaaaggctccgtggttctggcctacagtggcggcctggacacctcgtgcatcctcgtgtggctgaaggaacaaggctatgacgtcattgcctatctggccaacattggccagaaggaagacttcgaggaagccaggaagaaggcactgaagcttggggccaaaaaggtgttcattgaggatgtcagcagggagtttgtggaggagttcatctggccggccatccagtccagcgcactgtatgaggaccgctacctcctgggcacctctcttgccaggccctgcatcgcccgcaaacaagtggaaatcgcccagcgggagggggccaagtatgtgtcccacggcgccacaggaaaggggaacgatcaggtccggtttgagctcagctgctactcactggccccccagataaaggtcattgctccctggaggatgcctgaattctacaaccggttcaagggccgcaatgacctgatggagtacgcaaagcaacacgggattcccatcccggtcactcccaagaacccgtggagcatggatgagaacctcatgcacatcagctacgaggctggaatcctggagaaccccaagaaccaagcgcctccaggtctctacacgaagacccaggacccagccaaagcccccaacacccctgacattctcgagatcgagttcaaaaaaggggtccctgtgaaggtgaccaacgtcaaggatggcaccacccaccagacctccttggagctcttcatgtacctgaacgaagtcgcgggcaagcatggcgtgggccgtattgacatcgtggagaaccgcttcattggaatgaagtcccgaggtatctacgagaccccagcaggcaccatcctttaccatgctcatttagacatcgaggccttcaccatggaccgggaagtgcgcaaaatcaaacaaggcctgggcttgaaatttgctgagctggtgtataccggtttacggcctagccctgagtgtgaatttgtccgccactgcatcgccaagtcccaggagcgagtggaagggaaagtgcaggtgtccgtcctcaagggccaggtgtacatcctcggccgggagtccccactgtctctctacaatgaggagctggtgagcatgaacgtgcagggtgattatgagccaactgatgccaccgggttcatcaacatcaattccctcaggctgaaggaatatcatcgtctccagagcaaggtcactgccaaatag

SEQ ID NO: 2 - DNA, 1395 bp, Homo sapiens
atggcctcggagagtgggaagctttggggtggccggtttgtgggtgcagtggaccccatcatggagaagttcaacgcgtccattgcctacgaccggcacctttgggaggtggatgttcaaggcagcaaagcctacagcaggggcctggagaaggcagggctcctcaccaaggccgagatggaccagatactccatggcctagacaaggtggctgaggagtgggcccagggcaccttcaaactgaactccaatgatgaggacatccacacagccaatgagcgccgcctgaaggagctcattggtgcaacggcagggaagctgcacacgggacggagccggaatgaccaggtggtcacagacctcaggctgtggatgcggcagacctgctccacgctctcgggcctcctctgggagctcattaggaccatggtggatcgggcagaggcggaacgtgatgttctcttcccggggtacacccatttgcagagggcccagcccatccgctggagccactggattctgagccacgccgtggcactgacccgagactctgagcggctgctggaggtgcggaagcggatcaatgtcctgcccctggggagtggggccattgcaggcaatcccctgggtgtggaccgagagctgctccgagcagaactcaactttggggccatcactctcaacagcatggatgccactagtgagcgggactttgtggccgagttcctgttctggcgttcgctgtgcatgacccatctcagcaggatggccgaggacctcatcctctactgcaccaaggaattcagcttcgtgcagctctcagatgcctacagcacgggaagcagcctgatgccccagaagaaaaaccccgacagtttggagctgatccggagcaaggctgggcgtgtgtttgggcggtgtgccgggctcctgatgaccctcaagggacttcccagcacctacaacaaagacttacaggaggacaaggaagctgtgtttgaagtgtcagacactatgagtgccgtgctccaggtggccactggcgtcatctctacgctgcagattcaccaagagaacatgggacaggctctcagccccgacatgctggccactgaccttgcctattacctggtccgcaaagggatgccattccgccaggcccacgaggcctccgggaaagctgtgttcatggccgagaccaagggggtcgccctcaaccagctgtcactgcaggagctgcagaccatcagccccctgttctcgggcgacgtgatctgcgtgtgggactacgggcacagtgtggagcagtatggtgccctgggcggcactgcgcgctccagcgtcgactggcagatccgccaggtgcgggcgctactgcaggcacagcaggcctag

SEQ ID NO: 3 - DNA, 21 bp, Artificial
  Primer
atgtccagcaaaggctccgtg

SEQ ID NO: 4 - DNA, 21 bp, Artificial
  Primer
ccgtgttgctttgcgtactcc

SEQ ID NO: 5 - DNA, 27 bp, Artificial
  Primer
ctccatatgtccagcaaaggctccgtg

SEQ ID NO: 6 - DNA, 27 bp, Artificial
  Primer
gaggcggccgctttggcagtgaccttg

SEQ ID NO: 7 - protein, 412 aa, Homo sapiens
MSSKGSVVLAYSGGLDTSCILVWLKEQGYDVIAYLANIGQKEDFEEARKKALKLGAKKVFIEDVSREFVEEFIWPAIQSSALYEDRYLLGTSLARPCIARKQVEIAQREGAKYVSHGATGKGNDQVRFELSCYSLAPQIKVIAPWRMPEFYNRFKGRNDLMEYAKQHGIPIPVTPKNPWSMDENLMHISYEAGILENPKNQAPPGLYTKTQDPAKAPNTPDILEIEFKKGVPVKVTNVKDGTTHQTSLELFMYLNEVAGKHGVGRIDIVENRFIGMKSRGIYETPAGTILYHAHLDIEAFTMDREVRKIKQGLGLKFAELVYTGLRPSPECEFVRHCIAKSQERVEGKVQVSVLKGQVYILGRESPLSLYNEELVSMNVQGDYEPTDATGFININSLRLKEYHRLQSKVTAK

SEQ ID NO: 8 - protein, 464 aa, Homo sapiens
MASESGKLWGGRFVGAVDPIMEKFNASIAYDRHLWEVDVQGSKAYSRGLEKAGLLTKAEMDQILHGLDKVAEEWAQGTFKLNSNDEDIHTANERRLKELIGATAGKLHTGRSRNDQVVTDLRLWMRQTCSTLSGLLWELIRTMVDRAEAERDVLFPGYTHLQRAQPIRWSHWILSHAVALTRDSERLLEVRKRINVLPLGSGAIAGNPLGVDRELLRAELNFGAITLNSMDATSERDFVAEFLFWRSLCMTHLSRMAEDLILYCTKEFSFVQLSDAYSTGSSLMPQKKNPDSLELIRSKAGRVFGRCAGLLMTLKGLPSTYNKDLQEDKEAVFEVSDTMSAVLQVATGVISTLQIHQENMGQALSPDMLATDLAYYLVRKGMPFRQAHEASGKAVFMAETKGVALNQLSLQELQTISPLFSGDVICVWDYGHSVEQYGALGGTARSSVDWQIRQVRALLQAQQA

SEQ ID NO: 9 - DNA, 21 bp, Artificial
  Primer
atggcctcggagagtgggaag

SEQ ID NO: 10 - DNA, 23 bp, Artificial
  Primer
tgaccacctggtcattccggctc